(19) United States
(12) Patent Application Publication
(10) Pub. No.: US 2013/0096531 A1
(43) Pub. Date: Apr. 18, 2013

(54) HYPODERMIC NEEDLE ASSEMBLY HAVING A TRANSITION HUB FOR ENHANCING FLUID DYNAMICS AND MICROSPHERE INJECTABILITY

(71) Applicants: Phillip A. Estepa, San Diego, CA (US); Reynaldo B. Halili, San Diego, CA (US); Christine P. Thai, San Diego, CA (US); Quanmin Chen, Germantown, MD (US); Ge Bai, Germantown, MD (US)

(72) Inventors: Phillip A. Estepa, San Diego, CA (US); Reynaldo B. Halili, San Diego, CA (US); Christine P. Thai, San Diego, CA (US); Quanmin Chen, Germantown, MD (US); Ge Bai, Germantown, MD (US)

(73) Assignee: Amylin Pharmaceuticals, Inc., San Diego, CA (US)

(21) Appl. No.: 13/520,965

(22) PCT Filed: Jan. 7, 2011
(86) PCT No.: PCT/US11/20567

§ 371 Date: Dec. 20, 2012

Related U.S. Application Data
(60) Provisional application No. 61/335,569, filed on Jan. 8, 2010.

(51) Int. Cl. A61M 5/32

(52) U.S. Cl. CPC A61M 5/3293
USPC 604/506; 604/240

(57) ABSTRACT

A hypodermic needle assembly and a method of delivering a microsphere drug. A hypodermic needle assembly includes an injection needle; an injection device; and a hub defining a cavity including a transition portion having a first end and a second end between the first end and an inlet of the injection needle and having a gradually decreasing diameter from the first end to the second end, wherein the hub is coupled between the injection needle and the injection device such that the first end is in fluidic communication with an outlet of the injection device and the second end is in fluidic communication with the inlet of the injection needle, wherein the diameter of the cavity at the first end is larger than an outlet diameter of the injection device, and wherein the diameter at the second end is substantially the same as an inlet diameter of the injection needle.

### CROSS-REFERENCE TO RELATED APPLICATION(S)

This application is a PCT International application of, and claims the benefit of and priority to, U.S. Patent Application No. 61/335,569 filed Jan. 8, 2010, the entire content of which is incorporated herein by reference.

### FIELD OF THE INVENTION

Aspects of embodiments of the present invention relate to hypodermic needle assemblies, and more particularly to transition hubs of hypodermic needle assemblies configured to prevent or reduce clogging.

### BACKGROUND

Hypodermic needle assemblies are commonly used for injecting medicinal drugs into patients and typically include a syringe for delivering the drug, a needle cannula including a passageway for injecting the drug into the patient, and a transition hub coupling the needle cannula to the syringe. Typically, medicinal drugs which are injectable through a hypodermic needle assembly are in a solution or mixture form and are highly fluidic. As such, conventional hypodermic needle assemblies are generally not designed to prevent or reduce clogging of the injected substance therein. Additionally, conventional hypodermic needle assemblies typically have a region characterized by a great amount of turbulence where the drug moves through the transition hub to the passageway of the needle cannula.

However, due to the turbulence-inducing transition region between the hub and the needle cannula, as well as large “dead spaces” in the cavity of the transition hub where flow is slow or stagnant, in the above-described conventional hypodermic needle assemblies, such assemblies are prone to clogging when utilized for viscous solutions or suspensions having large particles, such as microspheres. Therefore, a need exists for a hypodermic needle assembly configured to deliver viscous drug solutions or suspensions having particles, such as microspheres, while reducing or preventing clogging within the assembly.

### SUMMARY OF THE INVENTION

Aspects of embodiments of the present invention provide a hypodermic needle assembly configured to provide a smooth, preferably laminar, flow at a transition into a passageway of an injection needle to reduce or prevent clogging, such as when utilized for injecting a viscous drug solution or a drug containing particles, such as microspheres. Further aspects of embodiments of the present invention provide a hypodermic needle assembly having a transition hub cavity having a diameter that gradually decreases toward an outlet end, and a smooth transition between the transition hub cavity and an injection needle passageway, so as to enhance laminar flow of a pharmaceutical product delivered through the hypodermic needle assembly, reduce injection force (especially when delivering a product having relatively high viscosity and/or particle size), reduce stagnant areas within the transition hub cavity, and/or reduce or prevent clogging in the transition hub cavity and injection needle passageway.

According to one exemplary embodiment of the present invention, a hypodermic needle assembly includes an injection needle defining a passageway through the injection needle, the passageway having an inlet; an injection device defining a passageway through the injection device, the passageway having an outlet; and a hub defining a cavity in the hub, the cavity including a transition portion having a first end and a second end between the first end and the inlet of the passageway of the injection needle and having a gradually decreasing diameter from the first end to the second end, wherein the hub is coupled between the injection needle and the injection device such that the first end of the transition portion is in fluidic communication with the outlet of the passageway of the injection device and the second end of the transition portion is in fluidic communication with the inlet of the passageway of the injection needle, wherein the diameter of the cavity at the first end is larger than a diameter of the outlet of the passageway of the injection device, and wherein the diameter of the cavity at the second end is substantially the same as a diameter of the inlet of the passageway of the injection needle.

In one embodiment, the first end of the transition portion is proximate the outlet of the passageway of the injection device. In another embodiment, the first end of the transition portion is distal from the outlet of the passageway of the injection device.

In one embodiment, the diameter of the cavity decreases at a substantially constant rate from the first end to the second end.

In one embodiment, the hub includes a curved internal surface defining at least a portion of the transition portion of the cavity. The curved internal surface may include a first portion that is concave. The curved internal surface may further include a second portion between the first portion and the second end of the transition portion that is convex.

In one embodiment, the cavity of the hub further includes a needle receiving portion, and the injection needle is inserted in the needle receiving portion. In one embodiment, the cavity of the hub further includes an injection device receiving portion, and the injection device is inserted in the injection device receiving portion. The injection device may include a syringe.

In one embodiment, the inlet of the passageway of the injection needle is spaced apart from the second end of the transition portion.

According to another exemplary embodiment of the present invention, a method of delivering a microsphere drug to a patient utilizing a hypodermic needle assembly having an injection needle defining a passageway through the injection needle, an injection device defining a passageway through the injection device, and a transition hub defining a cavity in the transition hub including a transition portion having a first end and a second end between the first end and an inlet of the passageway of the injection needle and having a gradually decreasing diameter from the first end to the second end, the diameter at the second end being substantially the same as a diameter of the inlet of the passageway of the injection needle, the transition hub being coupled between the injection needle and the injection device such that the first end of the transition portion is in fluidic communication with the passageway of the injection device and the second end of the transition portion is in fluidic communication with the passageway of the injection needle, includes inserting an outlet end of the injection needle into the patient; and ejecting the microsphere drug through an outlet of the passageway of the injection device such that the microsphere drug passes through the cavity of the transition hub and subsequently through the passageway of the injection needle.

In one embodiment, the diameter of the cavity decreases at a substantially constant rate from the first end to the second end.

In one embodiment, the diameter of the cavity at the first end is substantially the same as a diameter of the outlet of the passageway of the injection device. In another embodiment, the diameter of the cavity at the first end is larger than a diameter of the outlet of the passageway of the injection device.

In one embodiment, the first end of the transition portion is proximate the outlet of the passageway of the injection device. The transition hub may include a curved internal surface defining at least a portion of the transition portion of the cavity.

Other features and advantages of embodiments of the present invention will become apparent from the following detailed description, taken in conjunction with the accompanying drawings which illustrate, by way of example, features and aspects of exemplary embodiments of the present invention.

### BRIEF DESCRIPTION OF THE DRAWINGS

The above and other features, aspects, and advantages of the present invention will become better understood with regard to the following description, appended claims, and accompanying drawings where:

FIG. 1 is an exploded sectional view of a hypodermic needle assembly according to an embodiment of the present invention;

FIG. 2 is a sectional view of the hypodermic needle assembly of FIG. 1 in an assembled state;

FIG. 3 is a sectional view of a hypodermic needle assembly according to another embodiment of the present invention;

FIG. 4 is a sectional view of a transition hub of the hypodermic needle assembly of FIG. 3;

FIG. 5 is a sectional view of a hypodermic needle assembly according to another embodiment of the present invention;

FIG. 6 is a sectional view of a transition hub of the hypodermic needle assembly of FIG. 5;

FIGS. 7A-7D are, respectively, analysis diagrams depicting strain rate distributions in the hypodermic needle assemblies of FIGS. 1, 3, and 5, and a conventional hypodermic needle assembly;

FIGS. 8A-8D are, respectively, further analysis diagrams depicting strain rate distributions in the hypodermic needle assemblies of FIGS. 1, 3, and 5, and a conventional hypodermic needle assembly;

FIGS. 9A, 9C, 9E, and 9G are, respectively, analysis diagrams depicting flow patterns in the hypodermic needle assemblies of FIGS. 1, 3, and 5, and a conventional hypodermic needle assembly;

FIGS. 9B, 9D, 9F, and 9H are, respectively, detail views of FIGS. 9A, 9C, 9E, and 9G showing a transition region of each of the hypodermic needle assemblies; and

FIGS. 10A-10D are, respectively, analysis diagrams depicting particle volume fraction distributions in the hypodermic needle assemblies of FIGS. 1, 3, and 5, and a conventional hypodermic needle assembly.

### DETAILED DESCRIPTION

In the following detailed description, certain exemplary embodiments of the present invention are shown and described, by way of illustration. As those skilled in the art would recognize, the described exemplary embodiments may be modified in various ways without departing from the spirit and scope of the present invention. Accordingly, the drawings and description are to be regarded as illustrative in nature, rather than restrictive.

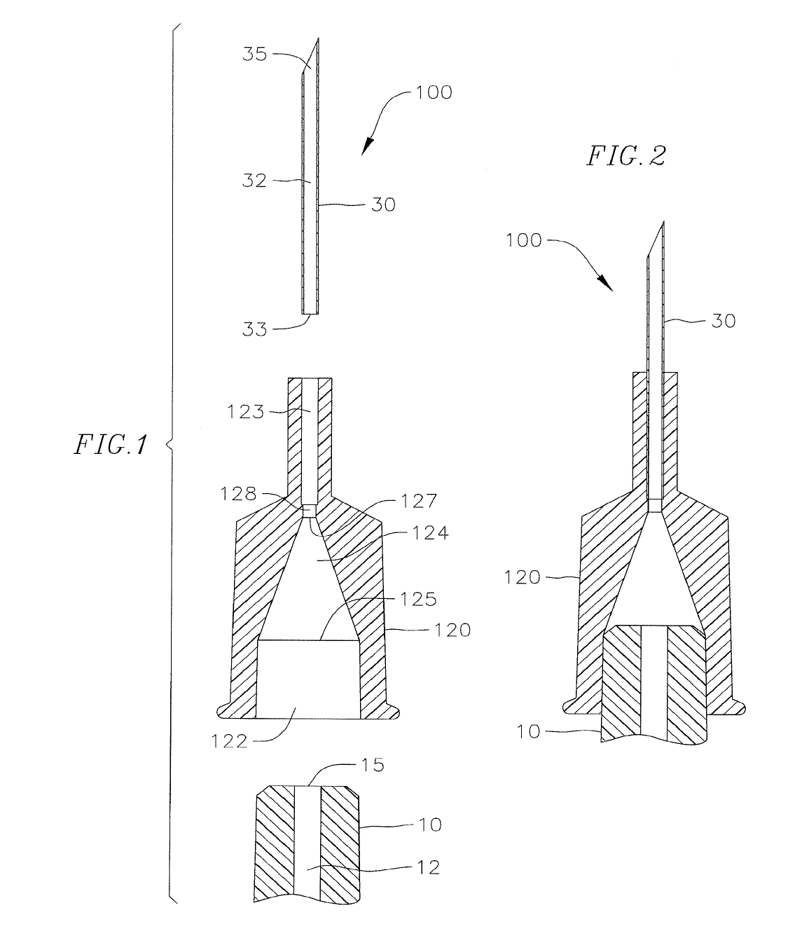

With reference to FIGS. 1 and 2, a hypodermic needle assembly 100 according to one exemplary embodiment of the present invention includes a syringe 10, a needle cannula 30, and a transition hub 120 coupled between the syringe 10 and the needle cannula 30. The hypodermic needle assembly 100 has a fluidic pathway from the syringe 10 to an outlet of the needle cannula 30 for delivering a pharmaceutical substance or other fluid substance contained in the syringe 10 through the transition hub 120 and the needle cannula 30.

The syringe 10, in embodiments of the present invention, may be any suitable syringe or other injection device configured to deliver a drug or other substance through the needle cannula 30 and into a patient. Further, while referred to herein as a syringe, the syringe 10, in embodiments of the present invention, may include any other suitable injection device or mechanism for delivering a drug or other substance, such as, but not limited to, an insulin injector or a pen injection device. With reference to FIGS. 1 and 2, only an outlet end of the syringe 10 is shown. The syringe 10 has a passageway 12 formed therein for providing a passage for the drug to move therethrough. Further, the passageway 12 has an outlet 15 at the outlet end of the syringe 10 through which the drug may exit from the syringe 10 and enter a cavity of the transition hub 120.

The needle cannula 30, in embodiments of the present invention, may be any suitable needle cannula or other injection needle (e.g., a hypodermic injection needle) configured to inject a drug or other substance into a patient. The needle cannula 30 has a passageway 32 formed therethrough along the length of the needle cannula 30 for providing a passage through which the drug may be moved. The passageway 32 has an inlet 33 at an inlet end of the needle cannula 30 through which the drug may be introduced into the passageway 32 from the transition hub 120 and an outlet 35 at an outlet end of the needle cannula 30 through which the drug may exit from the needle cannula 30. The needle cannula 30 may be formed of stainless steel or any other suitable material.

The transition hub 120, in one embodiment, has an internal cavity formed therein for providing a fluidic passageway between the outlet 15 of the syringe 10 and the inlet 33 of the needle cannula 30. In one embodiment, the internal cavity of the transition hub 120 includes a syringe receiving portion 122 (or injection device receiving portion) at one end for receiving the outlet end of the syringe 10 therein. Further, in one embodiment, the internal cavity of the transition hub 120 includes a needle receiving portion 123 at an end opposite the syringe receiving portion 122 for receiving an inlet end of the needle cannula 30 therein. In alternative embodiments, one or both of the syringe 10 and the needle cannula 30 may be coupled to the transition hub 120 in a manner other than by insertion into a portion of the internal cavity of the transition hub 120.

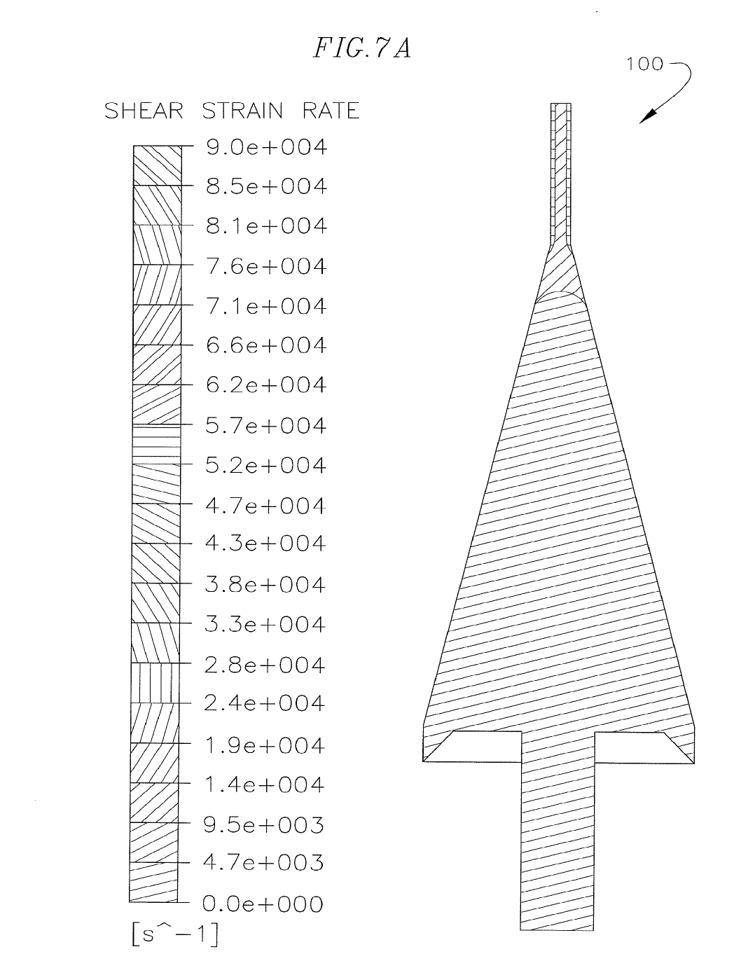

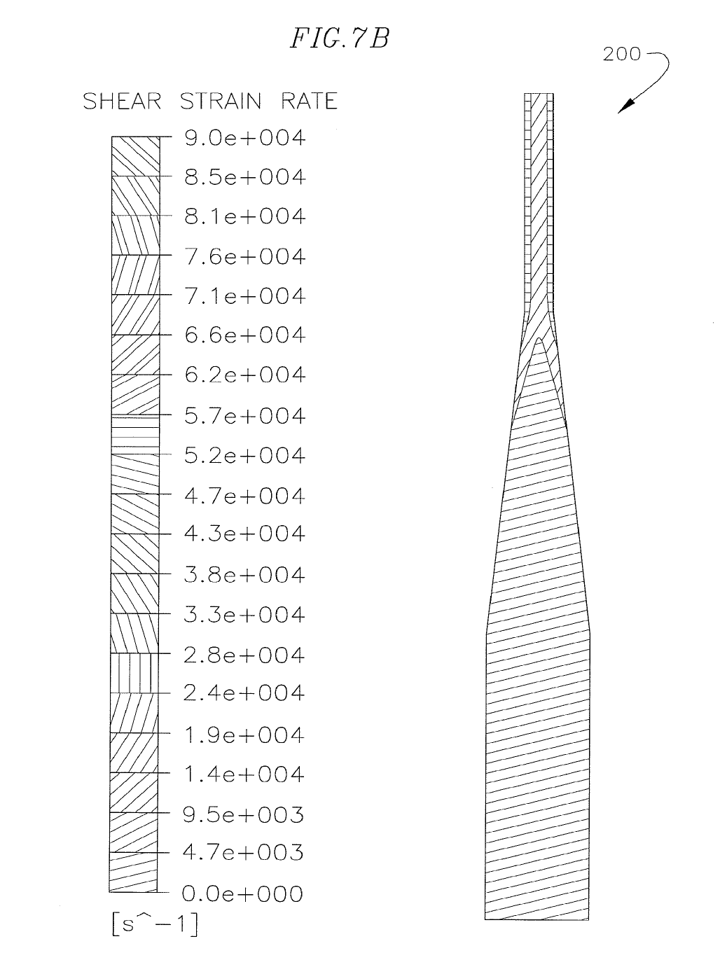

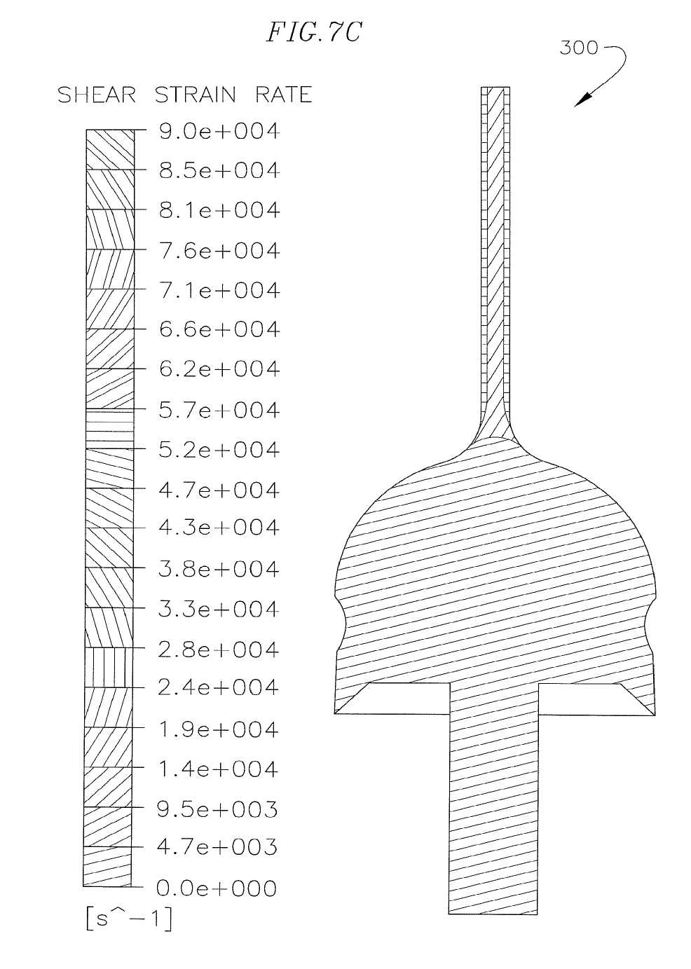

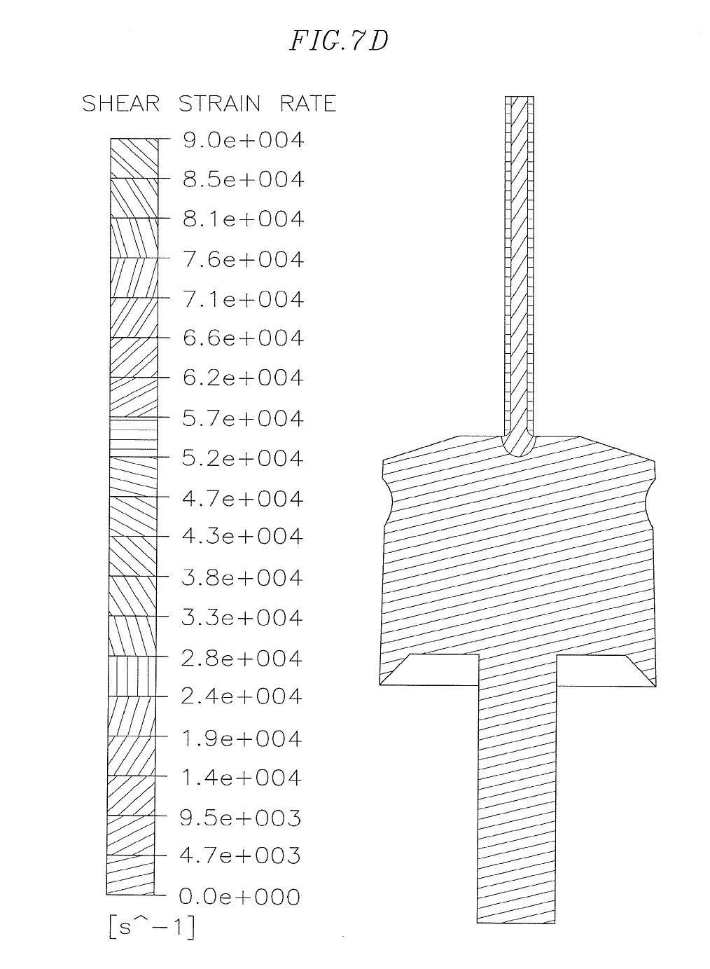

The internal cavity of the transition hub 120, in one embodiment, includes a transition portion 124. The transition portion 124 has a first end 125 in fluidic communication with the outlet 15 of the syringe 10 and a second end 127 that is between the first end 125 and the inlet 33 of the needle cannula 30 and in fluidic communication with the inlet 33 of the needle cannula 30. The transition portion 124, in one embodiment, has a gradually decreasing diameter from the first end 125 to the second end 127. In one embodiment, as shown in FIG. 1, the transition portion 124 has a funnel shape. Further, in one embodiment, the diameter of the transition portion 124 decreases at a constant or substantially constant rate from the first end 125 to the second end 127. The diameter of the internal cavity at the second end 127 of the transition portion 124, in one embodiment, is the same or substantially the same as an inner diameter of the inlet 33 of the needle cannula 30. Further, in one embodiment, as shown in FIGS. 1 and 2, the diameter of the internal cavity at the first end 125 is larger than a diameter of the outlet 15 of the syringe 10. The funnel-shaped configuration of the transition portion 124, as described further below in conjunction with FIGS. 7A-10D, enhances laminar flow for reducing or preventing clogging in the transition hub 120 and the needle cannula 30.

In one embodiment, the internal cavity of the transition hub 120 further includes an outlet portion 128 between the second end 127 of the transition portion 124 and the inlet 33 of the needle cannula 30. That is, the inlet 33 of the needle cannula 30 may be spaced apart from the second end 127 of the transition portion 124. The outlet portion 128, in one embodiment, has a diameter that is the same or substantially the same as the inner diameter of the inlet 33 of the needle cannula 30. In an alternative embodiment, the outlet portion 128 may be absent, such that the second end 127 of the transition portion 124 is proximate, rather than spaced apart from, the inlet 33 of the needle cannula 30. Additionally, although not shown in the embodiment of FIGS. 1 and 2, in other embodiments, the internal cavity of the transition hub 120 may include an inlet portion (e.g., an inlet portion similar to inlet portion 329 of FIG. 6) between the outlet 15 of the syringe 10 and the transition portion 124, such that the first end 125 of the transition portion 124 is spaced apart or distal from the outlet 15 of the syringe 10, rather than proximate the outlet 15 of the syringe 10 as shown in FIG. 2.

The transition hub 120, in one embodiment, is made of a plastic material, such as polypropylene, polyethylene, or polyurethane, and is formed by injection molding and/or insert molding, but alternatively may be made of any other plastic, a metal, or any other suitable material and may be formed by any other suitable process or device.

In one embodiment, as shown in FIG. 2, the syringe 10, the transition hub 120, and the needle cannula 30 are initially formed as three separate components, and the hypodermic needle assembly 100 is assembled by coupling these three components together. However, in alternative embodiments, two or three of the components may be integrally formed as a single component. For example, a needle cannula may be integrally formed with a transition hub having an internal cavity including the transition portion 124 (e.g., as a unitary molded structure). Further, a syringe may be integrally formed with a transition hub having an internal cavity including the transition portion 124. Additionally, in a further alternative embodiment, a syringe, a transition hub having an internal cavity including the transition portion 124, and a needle cannula may be integrally formed as a single unitary structure.

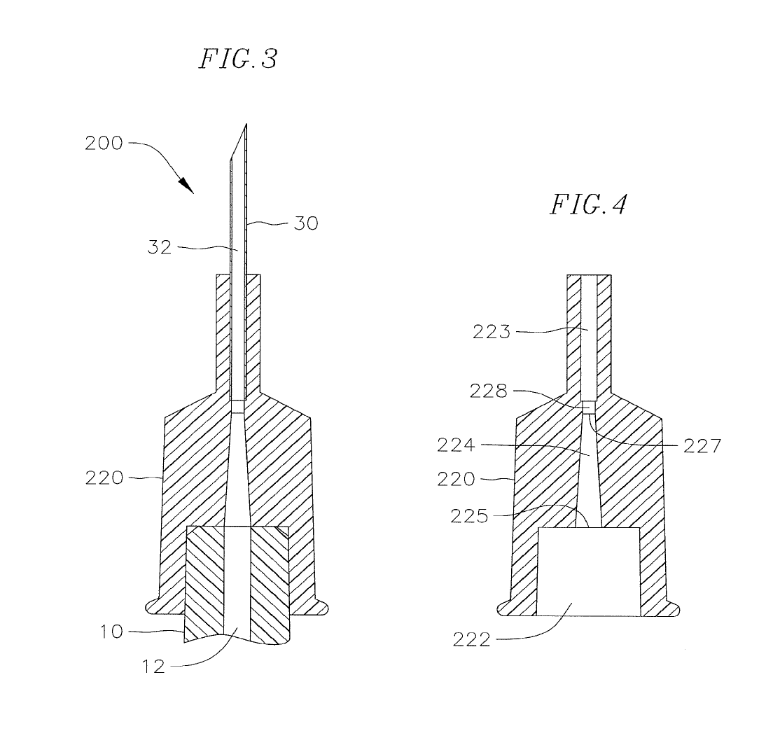

With reference to FIG. 3, a hypodermic needle assembly 200 according to another exemplary embodiment of the present invention includes a syringe 10, a needle cannula 30, and a transition hub 220 coupled between the syringe 10 and the needle cannula 30. The hypodermic needle assembly 200 has a fluidic pathway from the syringe 10 to an outlet of the needle cannula 30 for delivering a pharmaceutical substance or other fluid substance contained in the syringe 10 through the transition hub 220 and the needle cannula 30. The syringe 10 and the needle cannula 30 may be configured as described above with respect to the hypodermic needle assembly 100 and further description thereof will therefore be omitted.

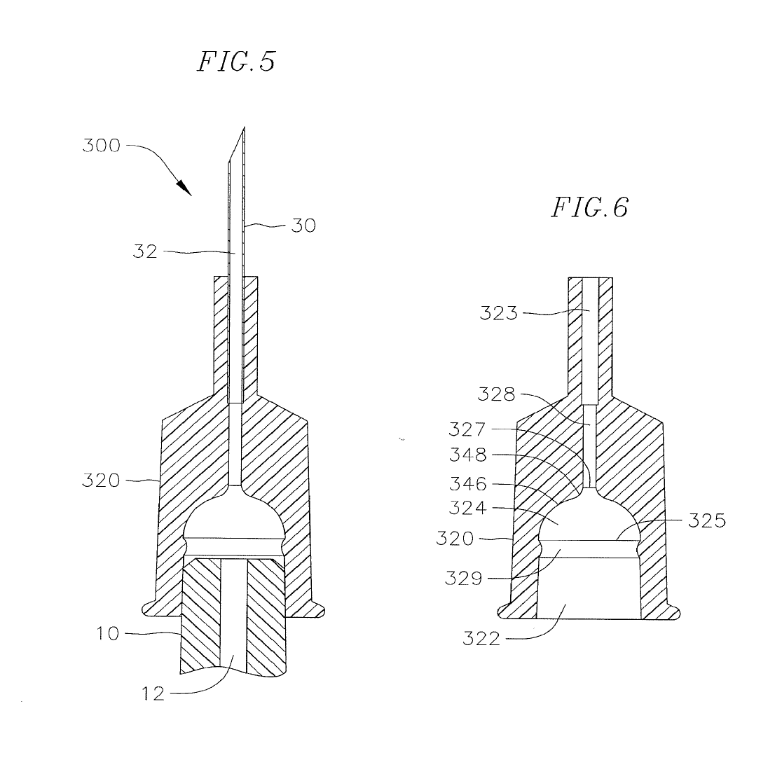

With reference to FIG. 4, the transition hub 220 has an internal cavity formed therein for providing a fluidic passageway between the outlet 15 of the syringe 10 and the inlet 33 of the needle cannula 30. In one embodiment, the internal cavity of the transition hub 220 includes a syringe receiving portion 222 (or injection device receiving portion) at one end for receiving the outlet end of the syringe 10 therein. Further, in one embodiment, the internal cavity of the transition hub 220 includes a needle receiving portion 223 at an end opposite the syringe receiving portion 222 for receiving an inlet end of the needle cannula 30 therein. However, as discussed above with respect to the hypodermic needle assembly 100, in alternative embodiments, one or both of the syringe 10 and the needle cannula 30 may be coupled to the transition hub 220 in a manner other than by insertion into a portion of the internal cavity of the transition hub 220.

The internal cavity of the transition hub 220, in one embodiment, includes a transition portion 224. The transition portion 224 has a first end 225 in fluidic communication with the outlet 15 of the syringe 10 and a second end 227 that is between the first end 225 and the inlet 33 of the needle cannula 30 and in fluidic communication with the inlet 33 of the needle cannula 30. The transition portion 224, in one embodiment, has a gradually decreasing diameter from the first end 225 to the second end 227. In one embodiment, as shown in FIG. 4, the transition portion 224 has a funnel shape. Further, in one embodiment, the diameter of the transition portion 224 decreases at a constant or substantially constant rate from the first end 225 to the second end 227. The diameter of the internal cavity at the second end 227 of the transition portion 224, in one embodiment, is the same or substantially the same as an inner diameter of the inlet 33 of the needle cannula 30. Further, in one embodiment, as shown in FIG. 3, the diameter of the internal cavity at the first end 225 is the same or substantially the same as a diameter of the outlet 15 of the syringe 10. That is, in one embodiment, the internal cavity of the transition hub 220 forms essentially a direct channel between the outlet 15 of the syringe 10 and the inlet 33 of the needle cannula 30. This direct channel configuration, as described further below in conjunction with FIGS. 7A-10D, enhances laminar flow for reducing or preventing clogging in the transition hub 220 and the needle cannula 30.

In one embodiment, the internal cavity of the transition hub 220 further includes an outlet portion 228 between the second end 227 of the transition portion 224 and the inlet 33 of the needle cannula 30. That is, the inlet 33 of the needle cannula 30 may be spaced apart from the second end 227 of the transition portion 224. The outlet portion 228, in one embodiment, has a diameter that is the same or substantially the same as the inner diameter of the inlet 33 of the needle cannula 30. In an alternative embodiment, the outlet portion 228 may be absent, such that the second end 227 of the transition portion 224 is proximate, rather than spaced apart from, the inlet 33 of the needle cannula 30. Additionally, although not shown in the embodiment of FIGS. 3 and 4, in other embodiments, the internal cavity of the transition hub 220 may include an inlet portion (e.g., an inlet portion similar to inlet portion 329 of FIG. 6) between the outlet 15 of the syringe 10 and the transition portion 224, such that the first end 225 of the transition portion 224 is spaced apart or distal from the outlet 15 of the syringe 10, rather than proximate the outlet 15 of the syringe 10 as shown in FIG. 3.

Similar to the transition hub 120 of the hypodermic needle assembly 100 described above, the transition hub 220, in one embodiment, is made of a plastic material, such as polypropylene, polyethylene, or polyurethane, and is formed by injection molding and/or insert molding. Alternatively, the transition hub 220 may be made of any other plastic, a metal, or any other suitable material and may be formed by any other suitable process or device.

In one embodiment, as shown in FIG. 3, the syringe 10, the transition hub 220, and the needle cannula 30 are initially formed as three separate components, and the hypodermic needle assembly 200 is assembled by coupling these three components together. However, as described above with respect to the hypodermic needle assembly 100, in alternative embodiments, two or three of the components may be integrally formed as a single component. For example, a needle cannula may be integrally formed with a transition hub having an internal cavity including the transition portion 224 (e.g., as a unitary molded structure). Further, a syringe may be integrally formed with a transition hub having an internal cavity including the transition portion 224. Additionally, in a further alternative embodiment, a syringe, a transition hub having an internal cavity including the transition portion 224, and a needle cannula may be integrally formed as a single unitary structure.

With reference to FIG. 5, a hypodermic needle assembly 300 according to another exemplary embodiment of the present invention includes a syringe 10, a needle cannula 30, and a transition hub 320 coupled between the syringe 10 and the needle cannula 30. The hypodermic needle assembly 300 has a fluidic pathway from the syringe 10 to an outlet of the needle cannula 30 for delivering a pharmaceutical substance or other fluid substance contained in the syringe 10 through the transition hub 320 and the needle cannula 30. The syringe 10 and the needle cannula 30 may be configured as described above with respect to the hypodermic needle assembly 100 and further description thereof will therefore be omitted.

With reference to FIG. 6, the transition hub 320 has an internal cavity formed therein for providing a fluidic passageway between the outlet 15 of the syringe 10 and the inlet 33 of the needle cannula 30. In one embodiment, the internal cavity of the transition hub 320 includes a syringe receiving portion 322 (or injection device receiving portion) at one end for receiving the outlet end of the syringe 10 therein. Further, in one embodiment, the internal cavity of the transition hub 320 includes a needle receiving portion 323 at an end opposite the syringe receiving portion 322 for receiving an inlet end of the needle cannula 30 therein. However, as discussed above with respect to the hypodermic needle assembly 100, in alternative embodiments, one or both of the syringe 10 and the needle cannula 30 may be coupled to the transition hub 320 in a manner other than by insertion into a portion of the internal cavity of the transition hub 320.

The internal cavity of the transition hub 320, in one embodiment, includes a transition portion 324. The transition portion 324 has a first end 325 in fluidic communication with the outlet 15 of the syringe 10 and a second end 327 that is between the first end 325 and the inlet 33 of the needle cannula 30 and in fluidic communication with the inlet 33 of the needle cannula 30. The transition portion 324, in one embodiment, has a gradually decreasing diameter from the first end 325 to the second end 327. In one embodiment, as shown in FIG. 6, the transition portion 324 is curved, such as a bell shape. Further, in one embodiment, as shown in FIG. 6, the curved surface includes a first curved portion 346 that is concave and a second curved portion 348 between the first curved portion 346 and the second end 327 that is convex. The diameter of the internal cavity at the second end 327 of the transition portion 324, in one embodiment, is the same or substantially the same as an inner diameter of the inlet 33 of the needle cannula 30. Further, in one embodiment, as shown in FIG. 5, the diameter of the internal cavity at the first end 325 is larger than a diameter of the outlet 15 of the syringe 10. The bell-shaped configuration of the transition portion 324, as described further below in conjunction with FIGS. 7A-10D, enhances laminar flow for reducing or preventing clogging in the transition hub 320 and the needle cannula 30.

In one embodiment, the internal cavity of the transition hub 320 further includes an outlet portion 328 between the second end 327 of the transition portion 324 and the inlet 33 of the needle cannula 30. That is, the inlet 33 of the needle cannula 30 may be spaced apart from the second end 327 of the transition portion 324. The outlet portion 328, in one embodiment, has a diameter that is the same or substantially the same as the inner diameter of the inlet 33 of the needle cannula 30. In an alternative embodiment, the outlet portion 328 may be absent, such that the second end 327 of the transition portion 324 is proximate, rather than spaced apart from, the inlet 33 of the needle cannula 30. Additionally, in one embodiment, the internal cavity of the transition hub 320 includes an inlet portion 329 between the outlet 15 of the syringe 10 and the transition portion 324, such that the first end 325 of the transition portion 324 is spaced apart or distal from the outlet 15 of the syringe 10. However, in an alternative embodiment, the inlet portion 329 may be absent, such that the first end 325 of the transition portion 324 is proximate, rather than spaced apart from, the outlet 15 of the syringe 10.

Similar to the transition hub 120 of the hypodermic needle assembly 100 described above, the transition hub 320, in one embodiment, is made of a plastic material, such as polypropylene, polyethylene, or polyurethane, and is formed by injection molding and/or insert molding. Alternatively, the transition hub 320 may be made of any other plastic, a metal, or any other suitable material and may be formed by any other suitable process or device.

In one embodiment, as shown in FIG. 5, the syringe 10, the transition hub 320, and the needle cannula 30 are initially formed as three separate components, and the hypodermic needle assembly 300 is assembled by coupling these three components together. However, as described above with respect to the hypodermic needle assembly 100, in alternative embodiments, two or three of the components may be integrally formed as a single component. For example, a needle cannula may be integrally formed with a transition hub having an internal cavity including the transition portion 324 (e.g., as a unitary molded structure). Further, a syringe may be integrally formed with a transition hub having an internal cavity including the transition portion 324. Additionally, in a further alternative embodiment, a syringe, a transition hub having an internal cavity including the transition portion 324, and a needle cannula may be integrally formed as a single unitary structure.

To assemble the hypodermic needle assembly 100, 200, 300 of the embodiments shown in FIGS. 2, 3, and 5, the transition hub 120, 220, 320 is coupled to the outlet end of the syringe 10 via a threaded connection, a press-fit, or any other suitable coupling device or mechanism. According to some embodiments, as shown in FIGS. 2, 3, and 5, the outlet end of the syringe 10 is inserted into the syringe receiving portion 122, 222, 322 of the internal cavity of the transition hub 120, 220, 320. The needle cannula 30, according to the embodiments shown in FIGS. 2, 3, and 5, is inserted (e.g., by a press-fit) into the needle receiving portion 123, 223, 323 of the internal cavity of the transition hub 120, 220, 320. However, in alternative embodiments, the needle cannula 30 may be coupled to the transition hub 120, 220, 320 via a threaded connection or any other suitable coupling device or mechanism.

In use, embodiments of a hypodermic needle assembly according to the present invention may be utilized for delivering a microsphere drug, or any other desired viscous substance, solution, suspension, or other substance. For example, the microsphere drug may include polymers such as biocompatible polymers which may be biodegradable or non-biodegradable polymers, or blends or copolymers thereof. A polymer is biocompatible if the polymer and any degradation products of the polymer are non-toxic to the recipient and also possess no significant deleterious or untoward effects on the recipient's body, such as a substantial immunological reaction at the injection site. A biodegradable composition will degrade or erode in vivo to form smaller units or chemical species. Degradation can result, for example, by enzymatic, chemical, and physical processes. Suitable biocompatible, biodegradable polymers may include, for example, poly(lactides), poly(glycolides), poly(lactide-co-glycolides), poly(lactic acid)s, poly(glycolic acid)s, polycarbonates, polyesteramides, polyanydrides, poly(amino acids), polyorthoesters, poly(dioxanone)s, poly(alkylene alkylate)s, copolymers or polyethylene glycol and polyorthoester, biodegradable polyurethane, blends thereof, and copolymers thereof. Suitable biocompatible, non-biodegradable polymers may include non-biodegradable polymers selected from the group consisting of polyacrylates, polymers of ethylene-vinyl acetates and other acyl substituted cellulose acetates, non-degradable polyurethanes, polystyrenes, polyvinylchloride, polyvinyl fluoride, poly(vinyl imidazole), chlorosulphonate polyolefins, polyethylene oxide, blends thereof, and copolymers thereof. Molecular weights for polymers delivered utilizing embodiments of the present invention may be determined by a person of ordinary skill in the art taking into consideration factors such as the desired polymer degradation rate, physical properties such as mechanical strength, end group chemistry, and rate of dissolution of polymer in solvent. For example, a molecular weight may be about 2,000 Daltons to about 2,000,000 Daltons. In one embodiment, the polymer is a biodegradable polymer or copolymer. In another embodiment, the polymer is a poly(lactide-co-glycolide) (hereinafter “PLG”) with a lactide:glycolide ratio of about 1:1 and a molecular weight of about 10,000 Daltons to about 90,000 Daltons. In another embodiment, the molecular weight of the PLG is about 30,000 Daltons to about 70,000 Daltons, such as about 50,000 to about 60,000 Daltons. Polymers may also be selected based upon the polymer's inherent viscosity. Suitable inherent viscosities include about 0.06 to 1.0 dL/g, such as about 0.2 to 0.6 dL/g and, in one embodiment, between about 0.3 to 0.5 dL/g. Polymers may be degradable in 3 to 4 weeks. Further, polymers may be from Alkermes, Inc. under the trade name MEDISORB, such as those sold as 5050 DL 3A or 5050 DL 4A. The microspheres may be in the shapes of spheres, film, pellets, cylinders, discs, or any other suitable shapes. The microspheres may have a size of 1000 microns or less, and may be from about 1 micron to about 180 microns in diameter. Further, microspheres delivered utilizing embodiments of the present invention may be non-living and/or may have properties (e.g., therapeutic properties) that are not substantially affected when subjected to turbulent flow, such as through a conventional hypodermic needle hub.

A method, in one embodiment, of delivering a microsphere drug to a patient includes inserting an outlet end of the needle cannula 30 into the patient and depressing, or otherwise activating, the syringe 10 or other injection device of the hypodermic needle assembly 100, 200, 300 to eject the microsphere drug through the outlet 15 of the passageway 12 of the syringe 10 such that the microsphere drug passes through the internal cavity of the transition hub 120, 220, 320 and subsequently through the passageway 32 of the needle cannula 30. The method, in one embodiment, may further include assembling the hypodermic needle assembly prior to ejecting the microsphere drug, such as according to the above description of the hypodermic needle assemblies 100, 200, 300. The method may be carried out utilizing any of the hypodermic needle assemblies 100, 200, 300 described above. Moreover, as discussed, the method may be performed to deliver a drug, a liquid (e.g., a viscous liquid), a solution, a suspension, or any other desired substance other than one containing microspheres.

With reference to FIGS. 7A-7D, strain rate distributions in the hypodermic needle assemblies 100, 200, 300 described above, as well as in a conventional hypodermic needle assembly, are compared via a computational fluid dynamics (CFD) simulation. FIGS. 7A-7D are schematics of the CFD Simulations found in Ser. No. 61/335,569, incorporated by reference herein. In the simulation of FIGS. 7A-7D, each of the hypodermic needle assemblies includes a 25G needle, and the flow direction through the hypodermic needle assemblies is from bottom to top in the drawings. Strain rate values are represented by cross-hatching differences in the drawings, and as depicted in the drawings, the greatest strain rates occur near the entrance of the needle passageway and within the needle passageway. Further, with reference to Table 1 below, the hypodermic needle assembly 200 exhibits the lowest maximum strain rate, while the hypodermic needle assemblies 100 and 300 also exhibit a lower maximum strain rate than the conventional hypodermic needle assembly.

TABLE 1

| | Maximum Strain |
|---|---|
| Hypodermic Needle Assembly | Rate (1/s) |
| Hypodermic needle assembly 100 | 80,678 |
| (Funnel-shaped transition portion) | |
| Hypodermic needle assembly 200 | 76,313 |
| (Direct channel transition portion) | |
| Hypodermic needle assembly 300 | 79,531 |
| (Bell-shaped transition portion) | |
| Conventional needle assembly | 88,513 |
| (see FIG. 7D) | |

The conventional hypodermic needle assembly of FIG. 7D includes a transition hub having a cavity that is characterized by a generally cylindrical shape. That is, as shown in the drawing, the cavity has an inlet end having a diameter that is greater than a diameter of the outlet of the syringe passageway and an outlet end having a diameter that is greater than a diameter of the inlet of the needle passageway. Further, the diameter of the cavity is substantially constant from the inlet end to a location proximate the outlet end. That is, the conventional hypodermic needle assembly has a sharp reduction in the transition hub cavity diameter and a stepped transition between the transition hub cavity and the needle passageway. As such, the transition from the cavity of the transition hub to the passageway of the needle cannula is characterized by more turbulent flow and greater injection forces than in the hypodermic needle assembly according to embodiments of the present invention. Further, while injection forces required for delivering a drug through the hypodermic needle assembly according to embodiments of the present invention are generally lower than that of the conventional hypodermic needle assembly due to a more laminar flow being provided, the difference between the injection forces of the hypodermic needle assembly according to embodiments of the present invention and that of the conventional hypodermic needle assembly becomes greater as a function of the drug being more viscous and/or containing microspheres or other particles having larger size.

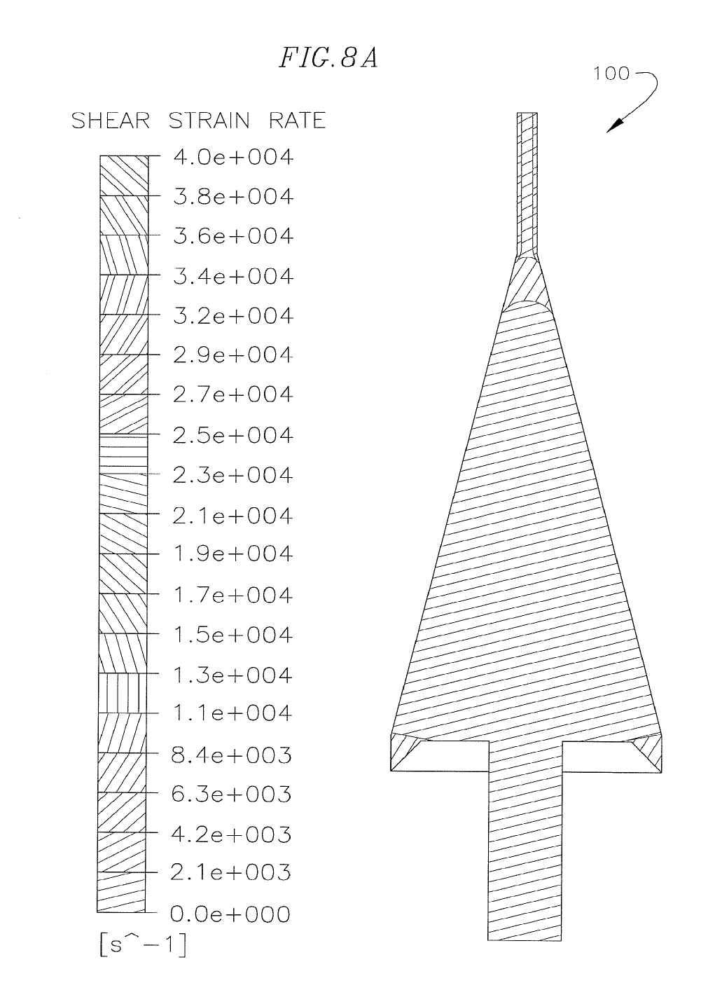

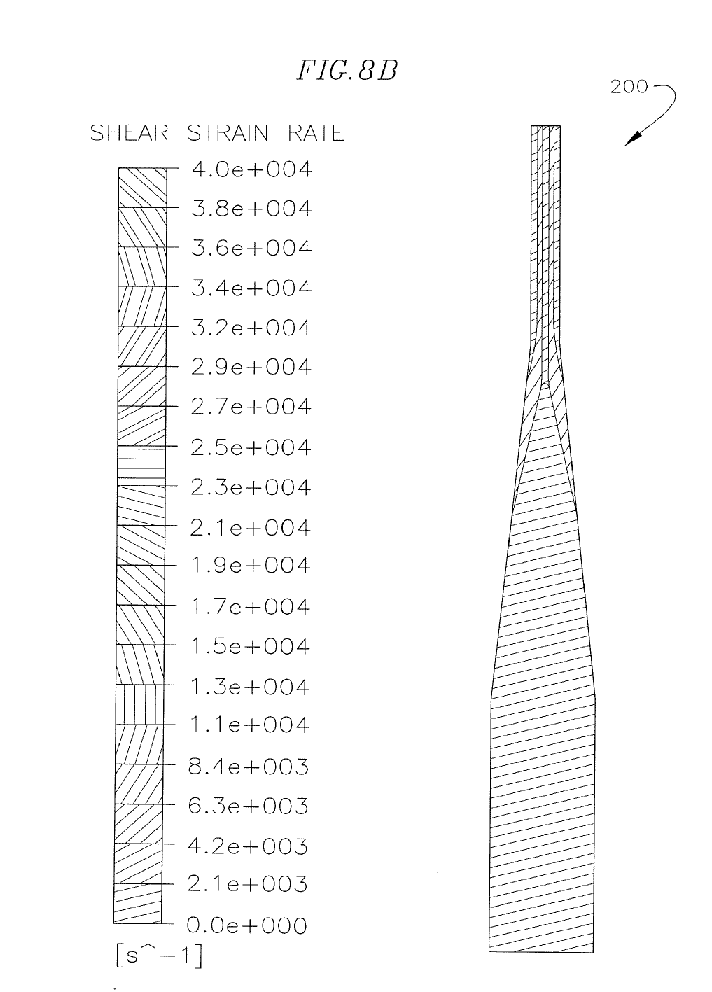

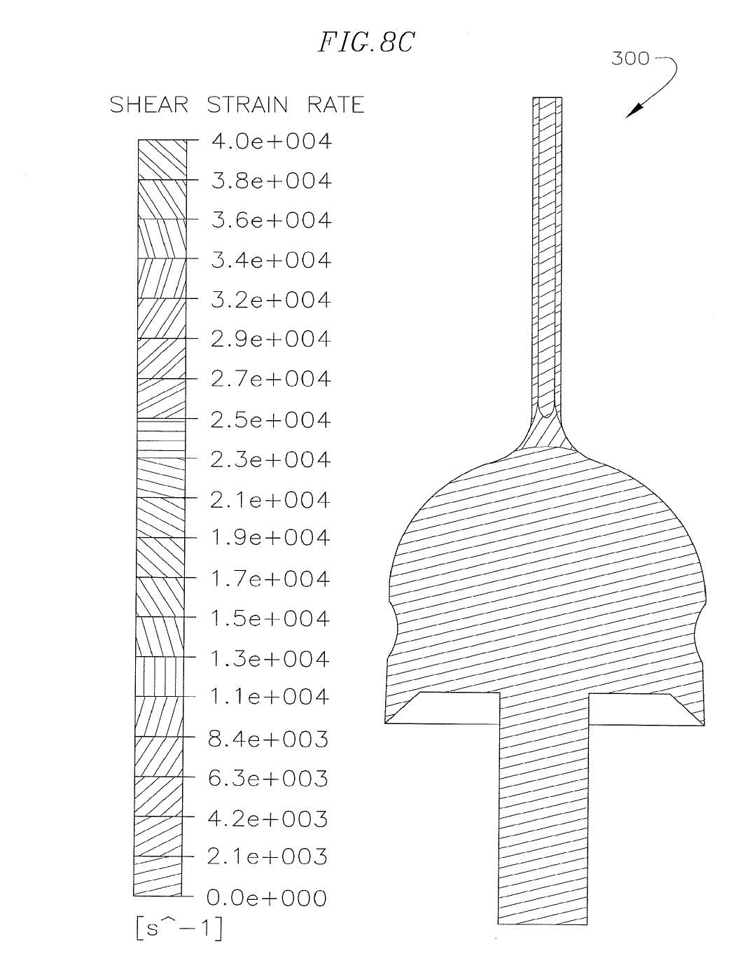

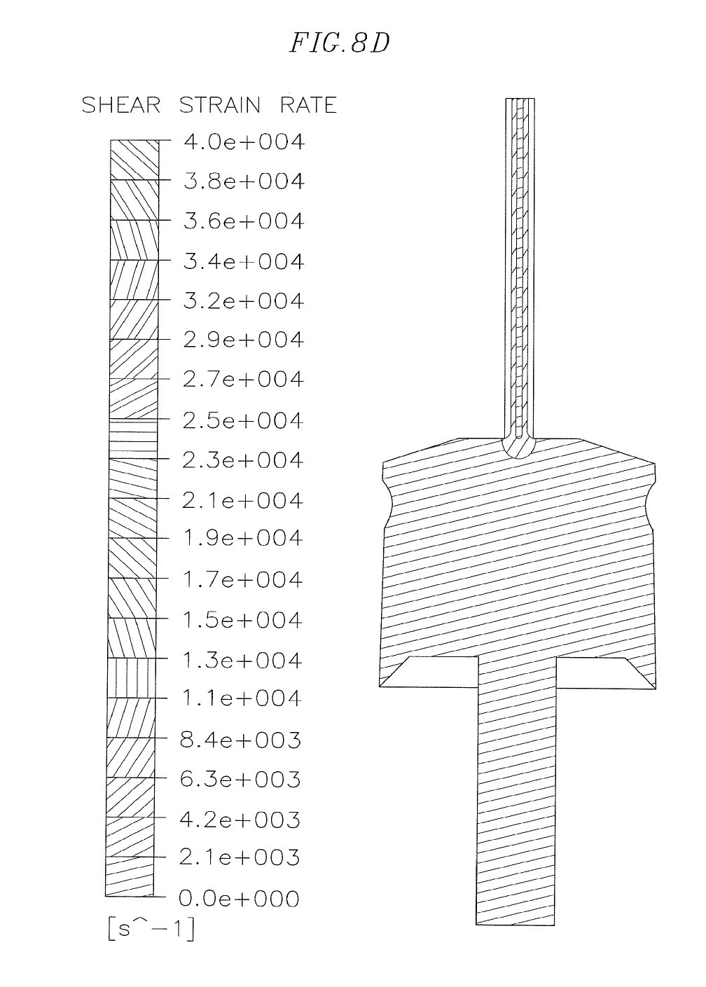

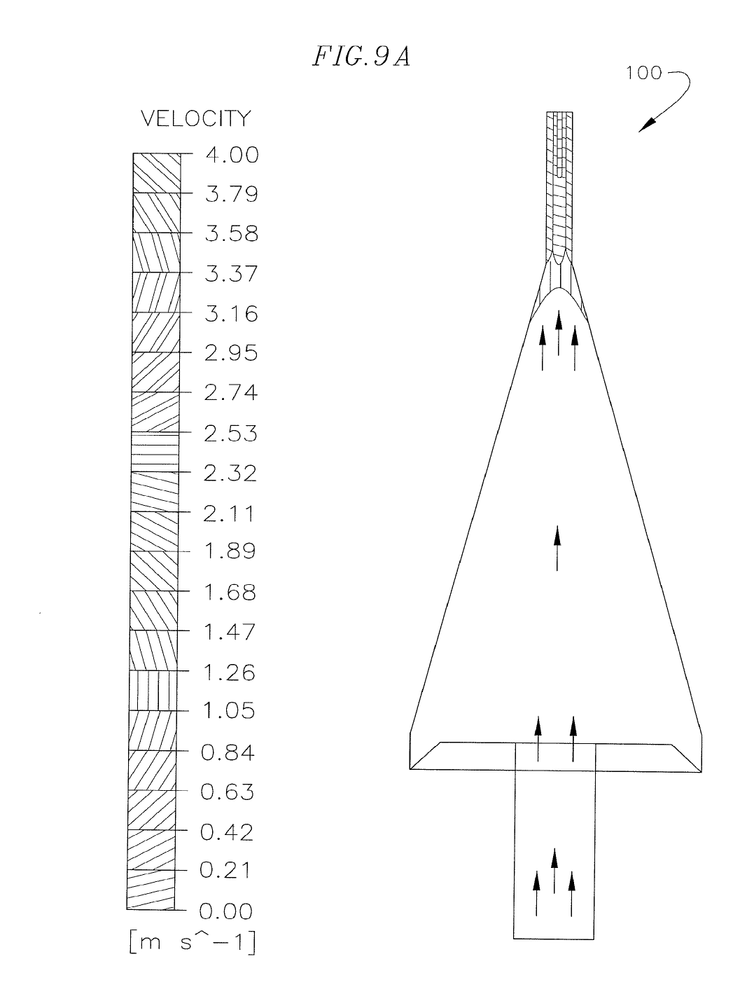

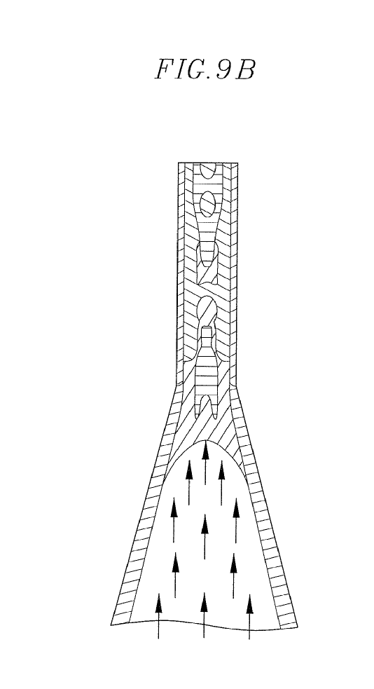

With reference to FIGS. 8A-8D, strain rate distributions in the hypodermic needle assemblies 100, 200, 300, as well as in the conventional hypodermic needle assembly described above, are compared via a CFD simulation. FIGS. 8A-8D are schematics of the CFD simulations found in Ser. No. 61/335,569, incorporated by reference herein. The strain rate distributions of FIGS. 8A-8D are similar to those shown in FIGS. 7A-7D except that each of the hypodermic needle assemblies includes a 23G needle instead of a 25G needle. The flow direction through the hypodermic needle assemblies is from bottom to top in the drawings. As illustrated in FIGS. 8A-8D, the greatest strain rates in each of the hypodermic needle assemblies occur near the entrance of the needle passageway and within the needle passageway. Further, with reference to Table 2 below, the hypodermic needle assembly 200 exhibits the lowest maximum strain rate, while the hypodermic needle assemblies 100 and 300 also exhibit a lower maximum strain rate than the conventional hypodermic needle assembly.

TABLE 2

| | Maximum Strain |
|---|---|
| Hypodermic Needle Assembly | Rate (1/s) |
| Hypodermic needle assembly 100 | 34,185 |
| (Funnel-shaped transition portion) | |
| Hypodermic needle assembly 200 | 32,250 |
| (Direct channel transition portion) | |
| Hypodermic needle assembly 300 | 37,751 |
| (Bell-shaped transition portion) | |
| Conventional needle assembly | 38,953 |
| (see FIG. 8D) | |

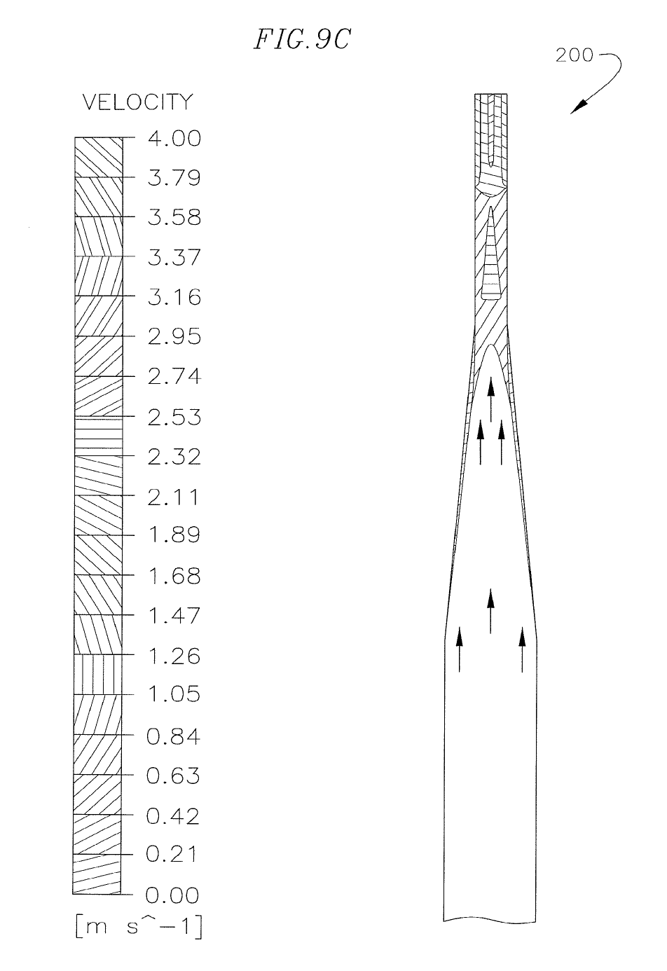

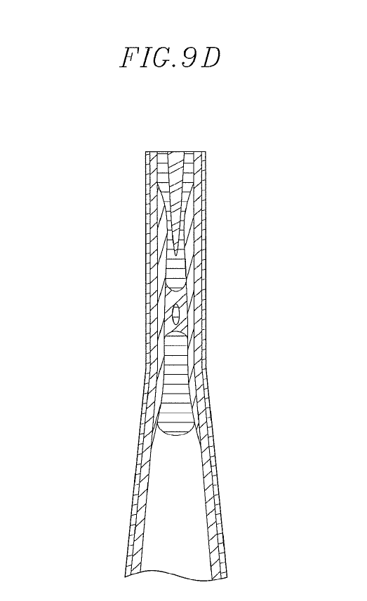

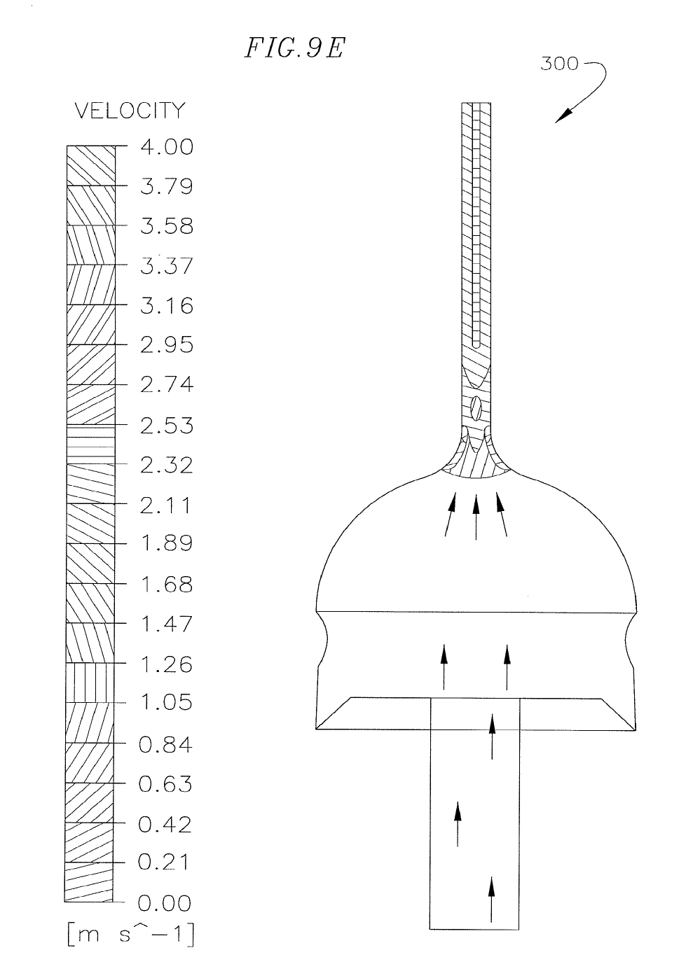

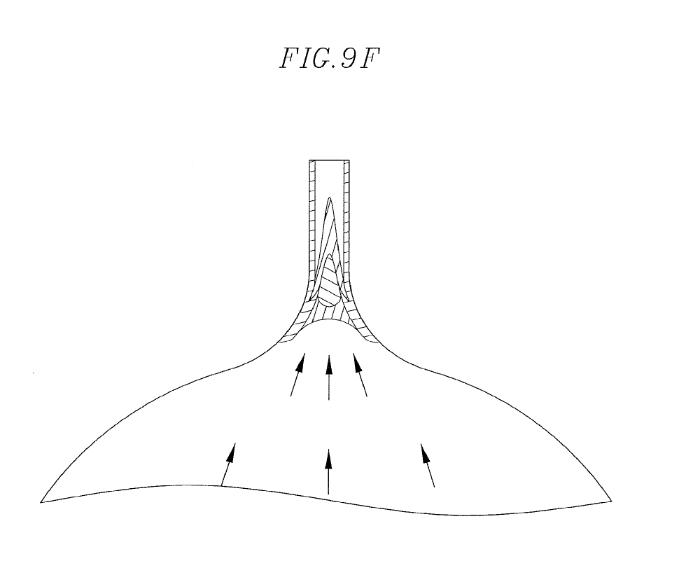

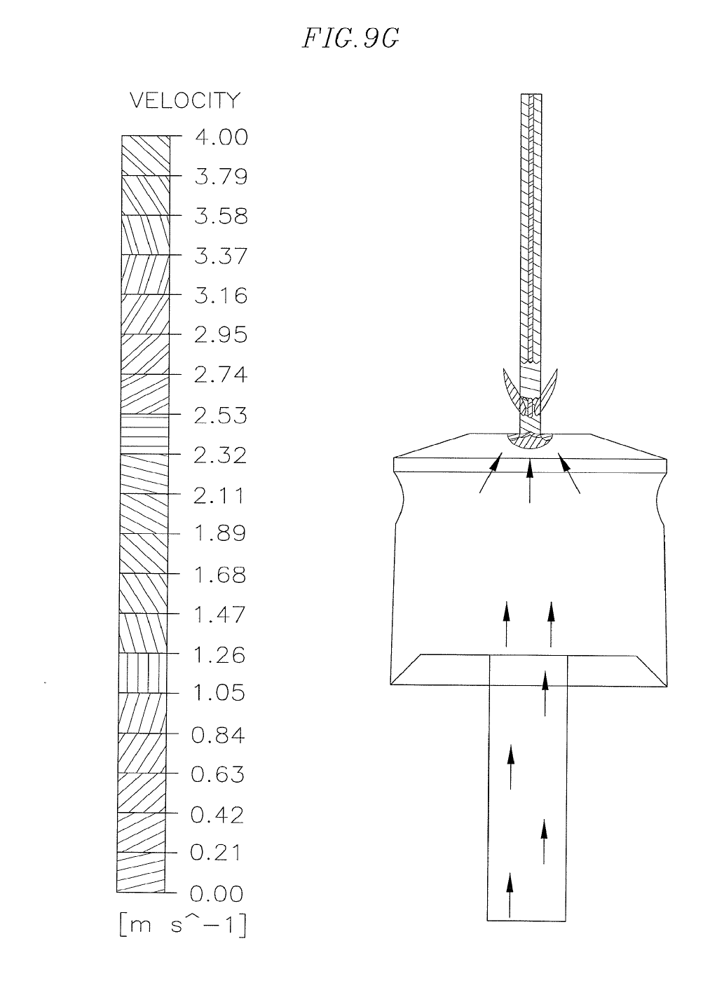

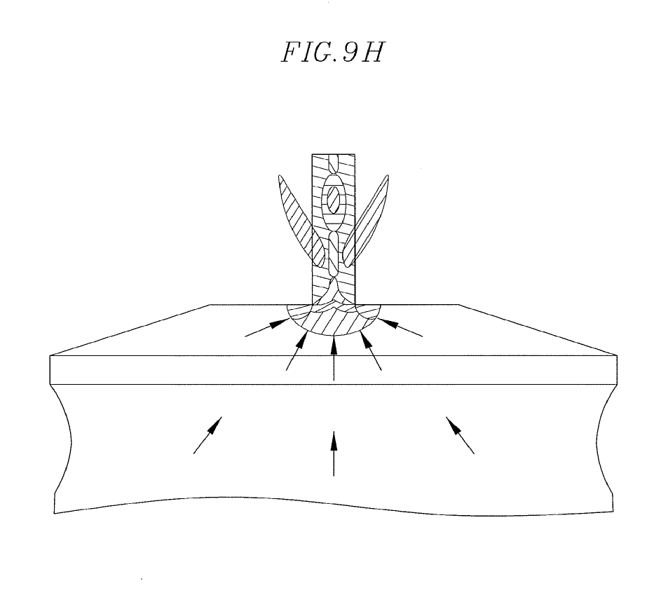

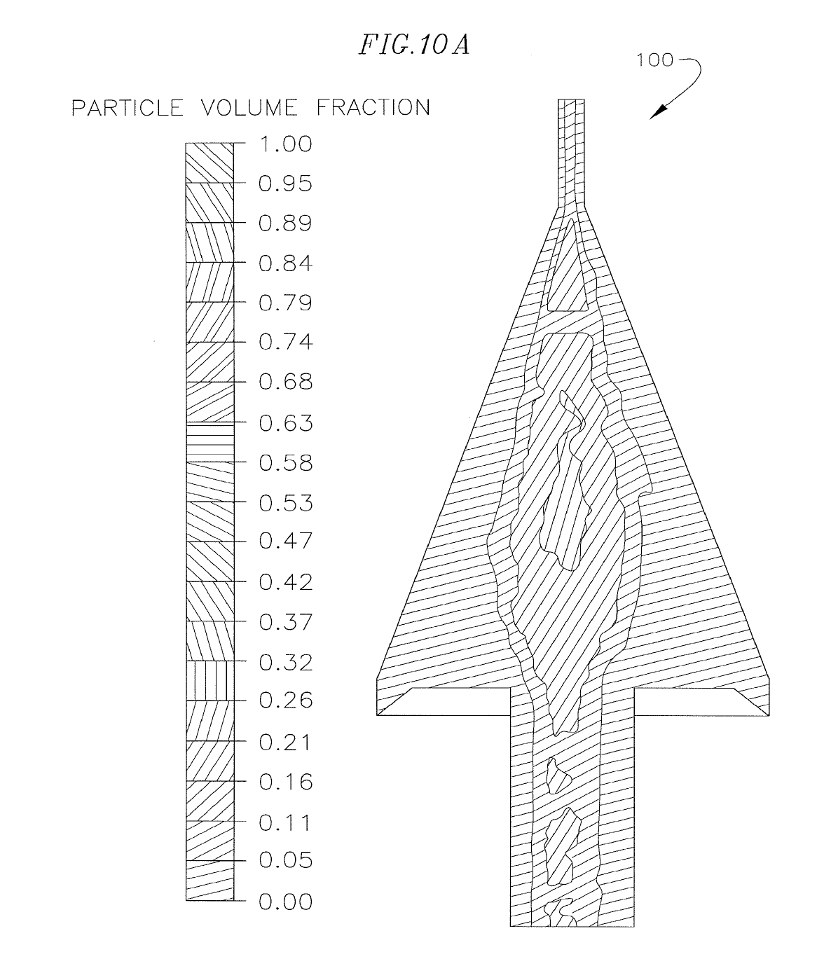

With reference to FIGS. 9A-9H, flow patterns in the hypodermic needle assemblies 100, 200, 300, as well as in the conventional hypodermic needle assembly described above, are compared via a CFD simulation. FIGS. 9A-9H are schematics of the CFD simulations found in Ser. No. 61/335,569, incorporated by reference herein. Each of the hypodermic needle assemblies of the simulation includes a 25G needle, and the flow direction through the hypodermic needle assemblies is from bottom to top in the drawings. Flow directions are depicted in the drawings via arrows, and flow velocities are represented by cross-hatching differences. As shown in greater detail in FIGS. 9B, 9D, and 9F, the hypodermic needle assemblies 100, 200, 300 exhibit fewer flow paths crossing one another near the transition between the transition hub cavity outlet and the needle passageway inlet than does the conventional hypodermic needle assembly shown in detail in FIG. 9H. As a result of fewer crossing flow paths near the transition of the hypodermic needle assemblies 100, 200, 300 of the present invention, particles, such as microspheres, suspended in the fluid traveling along the flow paths are less likely to collide and cause particle agglomerations that may clog the needle passageway or the transition hub cavity.

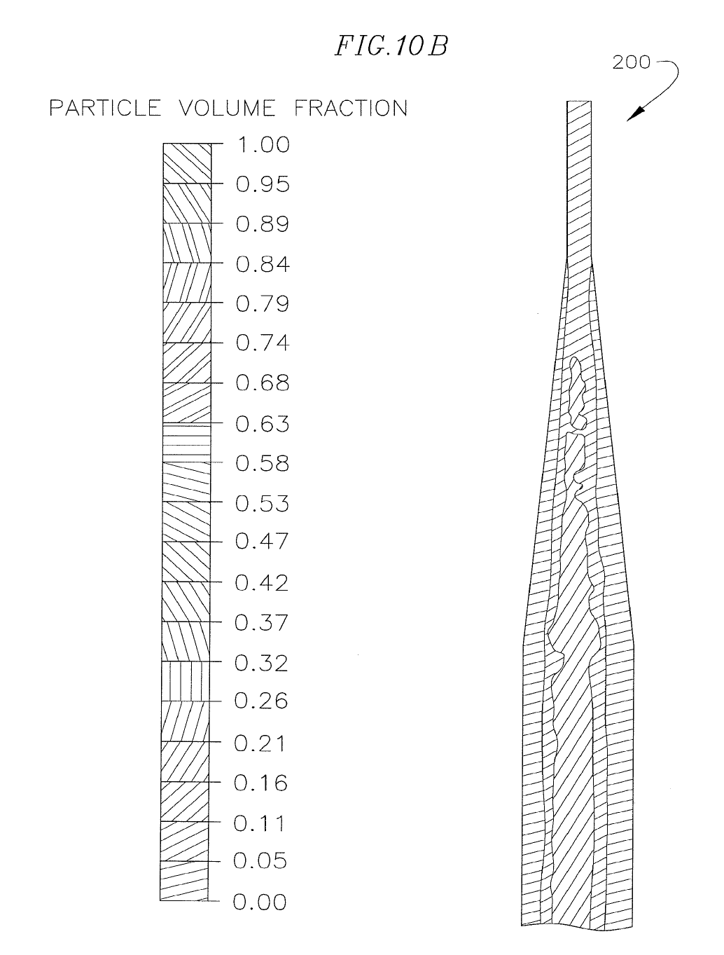

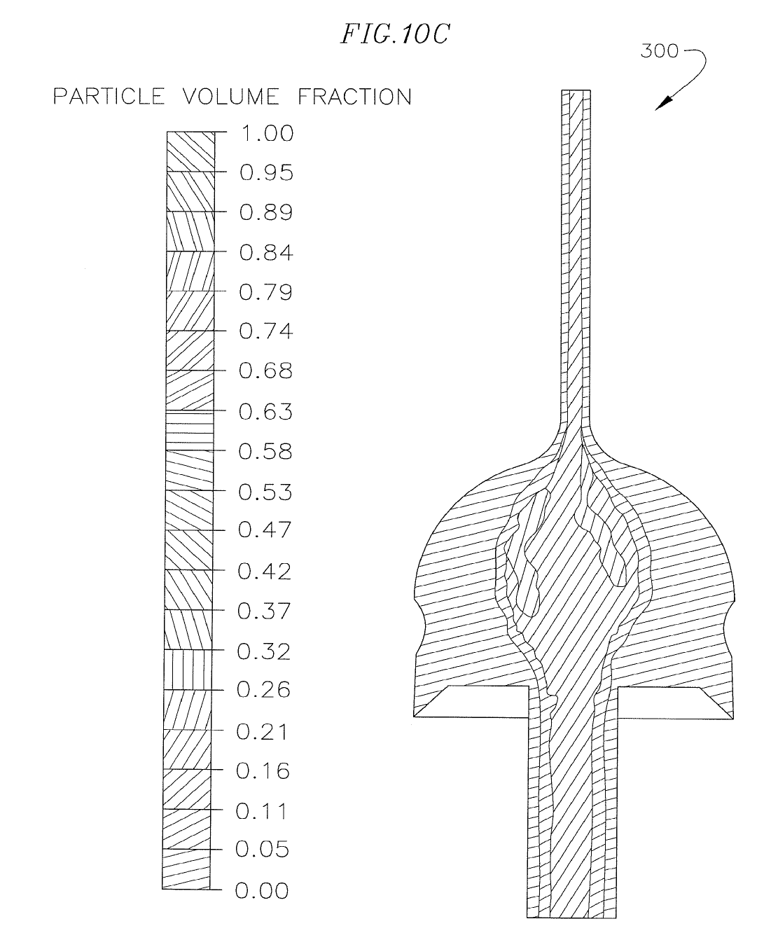

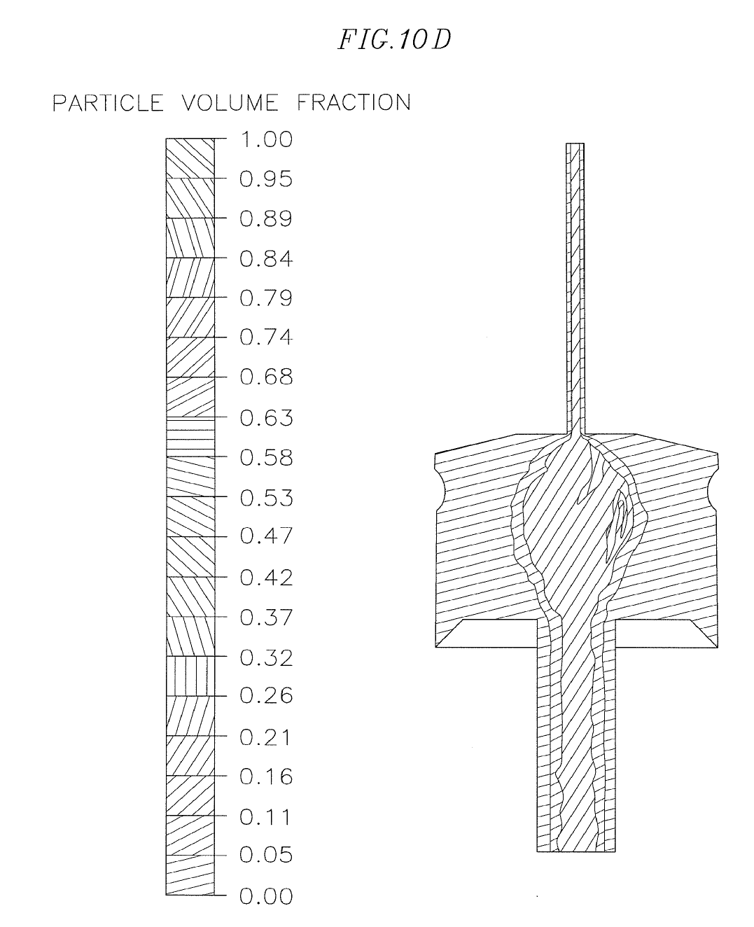

With reference to FIGS. 10A-10D, particle volume fraction distributions in the hypodermic needle assemblies 100, 200, 300, as well as in the conventional hypodermic needle assembly described above, are compared via a CFD simulation. FIGS. 10A-10D are schematics of the CFD simulations found in Ser. No. 61/335,569, incorporated by reference herein. Each of the hypodermic needle assemblies of the simulation includes a 25G needle, and the flow direction through the hypodermic needle assemblies is from bottom to top in the drawings. In FIGS. 10A-10D, regions having low particle volume fraction represent stagnant areas, or “dead space,” and are depicted in the drawings by cross-hatching type depicted near the bottom of the vertical bar. Particles in a suspension passing through each of the hypodermic needle assemblies tend to collect and accumulate in the stagnant areas, whereafter agglomerates of such particles are susceptible to breaking off, or avalanching, and clogging the passageway of the needle or the transition hub proximate the needle inlet. As illustrated in FIGS. 10A-10D, the hypodermic needle assembly 200 has the least stagnant area, while the hypodermic needle assemblies 100 and 300 also appear to have a smaller percentage of stagnant area than the conventional hypodermic needle assembly.

Although the drawings and accompanying description illustrate exemplary embodiments of a hypodermic needle assembly and a transition hub thereof, it will be apparent that the novel aspects of the hypodermic needle assembly and transition hub of the present invention may also be carried out by utilizing alternative structures, sizes, shapes, and/or materials in embodiments of the hypodermic needle assembly and transition hub of the present invention.

The preceding description has been presented with reference to various embodiments of the invention. Persons skilled in the art and technology to which this invention pertains will appreciate that alterations and changes in the described structures and methods of operation can be practiced without meaningfully departing from the principles, spirit, and scope of this invention.

What is claimed is:

1. A hypodermic needle assembly comprising:
an injection needle defining a passageway through the injection needle, the passageway having an inlet;
an injection device defining a passageway through the injection device, the passageway having an outlet; and
a hub defining a cavity in the hub, the cavity including a transition portion having a first end and a second end between the first end and the inlet of the passageway of the injection needle and having a gradually decreasing diameter from the first end to the second end, wherein the hub is coupled between the injection needle and the injection device such that the first end of the transition portion is in fluidic communication with the outlet of the passageway of the injection device and the second end of the transition portion is in fluidic communication with the inlet of the passageway of the injection needle,
wherein the diameter of the cavity at the first end is larger than a diameter of the outlet of the passageway of the injection device, and
wherein the diameter of the cavity at the second end is substantially the same as a diameter of the inlet of the passageway of the injection needle.

2. The hypodermic needle assembly of claim 1, wherein the first end of the transition portion is proximate the outlet of the passageway of the injection device.

3. The hypodermic needle assembly of claim 1, wherein the first end of the transition portion is distal from the outlet of the passageway of the injection device.

4. The hypodermic needle assembly of claim 1, wherein the diameter of the cavity decreases at a substantially constant rate from the first end to the second end.

5. The hypodermic needle assembly of claim 1, wherein the hub comprises a curved internal surface defining at least a portion of the transition portion of the cavity.

6. The hypodermic needle assembly of claim 5, wherein the curved internal surface comprises a first portion that is concave.

7. The hypodermic needle assembly of claim 6, wherein the curved internal surface further comprises a second portion between the first portion and the second end of the transition portion that is convex.

8. The hypodermic needle assembly of claim 1, wherein the cavity of the hub further includes a needle receiving portion, and the injection needle is inserted in the needle receiving portion.

9. The hypodermic needle assembly of claim 1, wherein the cavity of the hub further includes an injection device receiving portion, and the injection device is inserted in the injection device receiving portion.

10. The hypodermic needle assembly of claim 1, wherein the injection device comprises a syringe.

11. The hypodermic needle assembly of claim 1, wherein the inlet of the passageway of the injection needle is spaced apart from the second end of the transition portion.

12. A method of delivering a microsphere drug to a patient utilizing a hypodermic needle assembly having an injection needle defining a passageway through the injection needle, an injection device defining a passageway through the injection device, and a transition hub defining a cavity in the transition hub including a transition portion having a first end and a second end between the first end and an inlet of the passageway of the injection needle and having a gradually decreasing diameter from the first end to the second end, the diameter at the second end being substantially the same as a diameter of the inlet of the passageway of the injection needle, the transition hub being coupled between the injection needle and the injection device such that the first end of the transition portion is in fluidic communication with the passageway of the injection device and the second end of the transition portion is in fluidic communication with the passageway of the injection needle, the method comprising:
inserting an outlet end of the injection needle into the patient; and
ejecting the microsphere drug through an outlet of the passageway of the injection device such that the microsphere drug passes through the cavity of the transition hub and subsequently through the passageway of the injection needle.

13. The method of claim 12, wherein the diameter of the cavity decreases at a substantially constant rate from the first end to the second end.

14. The method of claim 12, wherein the diameter of the cavity at the first end is substantially the same as a diameter of the outlet of the passageway of the injection device.

15. The method of claim 12, wherein the diameter of the cavity at the first end is larger than a diameter of the outlet of the passageway of the injection device.

16. The method of claim 12, wherein the first end of the transition portion is proximate the outlet of the passageway of the injection device.

17. The method of claim 12, wherein the transition hub comprises a curved internal surface defining at least a portion of the transition portion of the cavity.
